(19) United States
(12) Patent Application Publication
(10) Pub. No.: US 2026/0294735 A1
(43) Pub. Date: Oct. 1, 2026

(54) Vibration Noise-to-Music Conversion System for Massage Device

(71) Applicant: Zhijian Li, Fuzhou (CN)

(72) Inventor: Zhijian Li, Fuzhou (CN)

(21) Appl. No.: 19/095,738

(22) Filed: Mar. 31, 2025

(51) Int. Cl. A61H 23/00; A61H 23/02; A61M 21/00; A61M 21/02

(52) U.S. Cl. CPC A61H 23/008; A61H 23/0254; A61M 21/02; A61H 2201/0157; A61H 2201/5005; A61H 2201/503; A61M 2021/0027

(57) ABSTRACT

A massage device includes a main body. Vibration motors are disposed within the main body. A controller is programmed to convert mechanical vibration noise generated by the massage motors into structured musical tones through frequency modulation, thereby resolving a conflict between therapeutic vibration effects and disruptive noise.

### FIELD

The following relates generally to a massage device, specifically for converting mechanical vibration noise generated by a massage motor into structured musical tones through frequency modulation, thereby replacing disruptive noise with therapeutic vibration effects.

### BACKGROUND

Traditional massage devices utilize eccentric rotor motors to generate vibrations for massaging a user. However, irregular collisions between the motor and structural components (e.g., straps) can produce harsh, non-musical noise. Existing solutions focus on passive noise reduction (e.g., damping materials), but fail to transform the noise into a structured auditory output that is auditorily pleasing for the user.

The following discloses certain improvements to overcome these problems and others.

### SUMMARY

The present disclosure addresses this by actively converting vibrations into musical tones using the disclosed control algorithm and the disclosed hardware design. The massage device includes the disclosed converter hardware and software in a wearable device, such as a backpack, vest, jacket, or any other suitable body. The term “body” as used herein refers to any suitable base device including, without limitation a bag, luggage, clothing, activewear, shoes, hats, glasses, belt or anything wearable or that can be carried.

According to one aspect of the present disclosure, a vibration noise-to-music conversion system for a massage device is disclosed. The conversion system includes a body; at least one massage device disposed on the body, the at least massage device including at least two vibration motors; and a controller programmed to execute a Sinusoidal Pulse Width Modulation (SPWM) algorithm, wherein the SPWM algorithm modulates vibration frequencies of the at least two vibration motors to correspond to musical notes. The controller synchronizes the vibration frequencies of the at least two vibration motors with a selected musical score to generate structured musical tones.

According to another aspect of the present disclosure, a user interface is configured for selecting musical scores and adjusting vibration amplitude.

According to another aspect of the present disclosure, the SPWM algorithm generates sinusoidal waveforms by varying pulse widths to match frequencies of musical scales.

According to another aspect of the present disclosure, the at least two vibration motors are independently driven by MOSFET-based circuits to enable zone-specific frequency control.

According to another aspect of the present disclosure, the user interface includes short-press buttons for cycling through musical presets; long-press buttons for adjusting vibration amplitude levels.

According to another aspect of the present disclosure, the controller is programmed to filter electromagnetic interference from motor driver circuits to maintain music signal fidelity.

According to another aspect of the present disclosure, the SPWM algorithm dynamically adjusts pulse duty cycles to control both frequency and amplitude.

According to another aspect of the present disclosure, a method for converting vibration noise into musical tones includes generating vibrations via at least three motors, modulating the vibration frequencies of the motors using an SPWM algorithm to align with musical note frequencies, receiving user input to select a musical score and adjust vibration intensity, and synchronizing the modulated frequencies across the motors to produce coherent musical output.

According to another aspect of the present disclosure, the method includes selecting, via a user interface, musical scores and adjusting vibration amplitude.

According to another aspect of the present disclosure, the SPWM algorithm generates sinusoidal waveforms by varying pulse widths to match frequencies of musical scales.

According to another aspect of the present disclosure, the at least three vibration motors are independently driven by MOSFET-based circuits to enable zone-specific frequency control.

According to another aspect of the present disclosure, the user interface includes short-press buttons for cycling through musical presets; long-press buttons for adjusting vibration amplitude levels.

According to another aspect of the present disclosure, the method includes filtering electromagnetic interference from motor driver circuits to maintain music signal fidelity.

According to another aspect of the present disclosure, the SPWM algorithm dynamically adjusts pulse duty cycles to control both frequency and amplitude.

According to another aspect of the present disclosure, a massage device that includes a main body. At least two vibration motors are disposed within the main body. A controller is programmed to synchronize vibration frequencies generated by the at least two vibration motors with a selected musical score to generate musical tones; and output the generated musical tones by sound generated by the vibrations of the motors.

According to another aspect of the present disclosure, the controller is configured to generate the structured musical tones by matching the vibration frequency of the vibration motors with a corresponding musical score from a table storing predetermined musical scores matched to predetermined vibration frequencies. Optionally an audio speaker can be included and can be controlled to output the musical score in a synchronized manner with the vibration motors.

According to another aspect of the present disclosure, the massage device includes a user interface that includes a short-press button that, when engaged by a user, is configured to control the controller to cycle through musical score presets until a user disengages with the short press-buttons to select the selected musical score, and a long-press button that, when engaged by a user is configured to control the controller to adjust vibration amplitude levels of the at least two vibration motors.

According to another aspect of the present disclosure, the controller is further programmed to adjust a duty cycle of the generated vibration frequencies to ensure the selected musical score continues to be output.

According to another aspect of the present disclosure, the main body comprises a backpack comprising a storage portion and two shoulder straps. The at least two vibration motors comprise three vibration motors, a first vibration motor being disposed in a first shoulder strap, a second vibration motor being disposed in a second shoulder strap, and a third vibration motor being disposed in the storage portion.

Advantageously, the present disclosure provides a massage device that converts massage vibrations (which can be unpleasant to listen to for a user of the massage device) generated by massage vibration motors into musical tones (which can be pleasant to listen to for the user).

Advantageously, the present disclosure provides a massage device that converts massage vibrations into musical tones with independently controlled massage vibration motors.

Advantageously, the present disclosure provides a massage device with selectable musical tones that are played by the massage device instead of massager vibrations.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 (including FIG. 1A and FIG. 1B) diagrammatically illustrates a plan view of massage device according to one aspect, with FIG. 1A and FIG. 1B showing the shoulder straps in a first position and a second position, respectively.

FIG. 2 diagrammatically illustrates electronics and other hardware of the massage device FIG. 1.

FIG. 3 shows a flowchart showing operations of an embodiment of a method performed by the massage device of FIG. 1.

FIGS. 4-9 show graphic representations of the operations of the method shown in FIG. 3.

### DETAILED DESCRIPTION

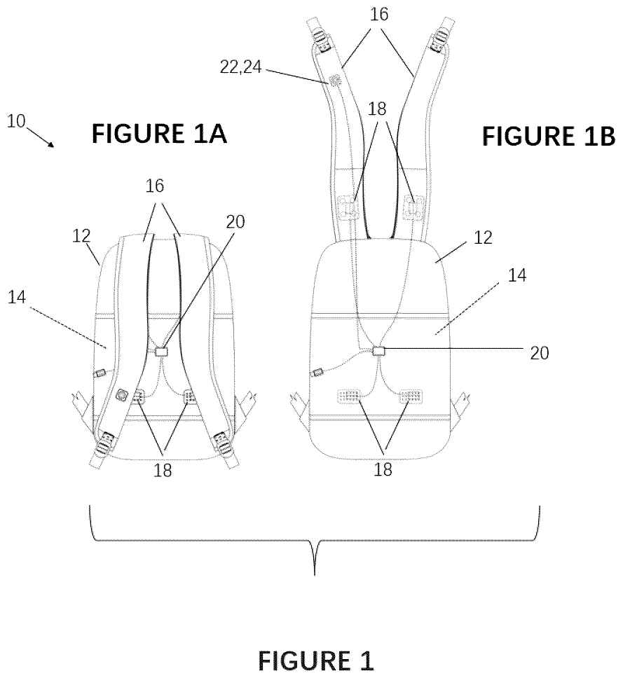

With reference now to FIG. 1, an embodiment of a massage device 10 is shown. As shown in FIG. 1, the massage device 10 can be configured as a backpack wearable by a user (not shown), but can also be configured as any suitable device engageable by the user (for example, a vest, a belt, a chair, and so forth). For brevity, the massage device 10 is primarily described herein as a backpack 10. As shown in FIG. 1, the backpack 10 can include a main body 12 with a storage portion 14 (i.e., an interior of the main body 12 for storing articles, shown in FIG. 1 with a dashed line), and shoulder straps 16 (two of which are shown in FIG. 1, although any suitable number of shoulder straps 16 can be included).

FIG. 1 also shows a plurality of (i.e., at least two) vibration motors 18 disposed within the main body 12, although any suitable number of vibration motors 18 can be included in the massage device 10. As shown in FIG. 1, three vibration motors 18 are included. A first vibration motor 18 is disposed in one of the shoulder straps 16, a second vibration motor 18 is disposed in the other shoulder strap 16, and a third vibration motor 18 is disposed in or adjacent the storage portion 14 in or adjacent a back panel of the main body 12 that lies adjacent a user's back when the body 12 is worn by the user (e.g., to overlie on a portion of the user's back when wearing the backpack 10). The vibration motors 18 are configured in a multi-zone layout to transmit vibrations through the shoulder straps 16 and the body 12 of the backpack 10. The vibration motors 18 are positioned on the backpack 10 to maximize resonance and minimize destructive interference. Each vibration motor 18 is configured to mechanically vibrate to massage a portion of the user's body that the vibration motor(s) 18 overlie. An electronic controller 20 such as a microcontroller is located in the body 12 and is electronically connected to each vibration motor 18 by respective wires or the like, and is also connected to one or more user input buttons 22, 24 configured to provide input to the controller 20 to provide instructions or input signals to the controller 20 to instruct the controller 20 to output control signals to the vibration motor(s) 18 to control operation of one or more parameters of the vibration motors 18 (e.g., intensity, duration, and so forth).

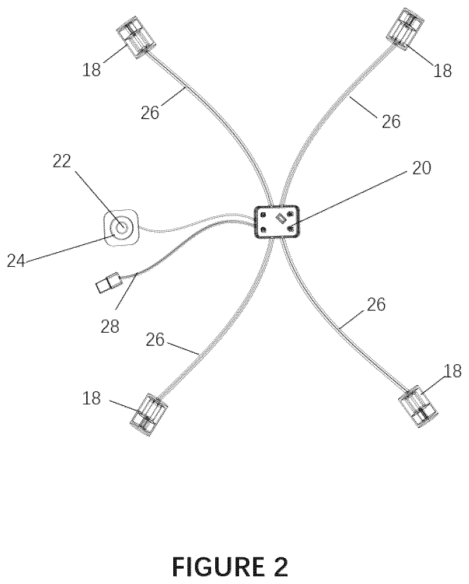

FIG. 2 shows electronics and associated hardware of the massage device 10 without showing the structure of the backpack 10 or other body 12. The electronic controller 20 is connected to each vibration motor 18 (four of which are shown in FIG. 2) via respective corresponding cables 26; although the controller 20 can be wirelessly connected to each vibration motor 18. Similarly, the controller 20 can be connected to the one or more controller input buttons 22, 24 by a cable 26. A power cord 28 is also connected to the controller and is configured to supply electrical power to the controller 20 and the vibration motors 18 from a power source connected to the cable 28 which can be a single use or rechargeable battery or other electrical power source stored in the storage portion 14 of the body 12.

The controller 20 includes typical computer processing components, such as a memory 32 and an electronic processor 34, for controlling operation of the vibration motors 18. The vibration motors 18 can produce vibrations with unpleasant sounds, which can be unpleasant for the user (wearer) of the massage device 10 to hear. The disclosed massage device 10 can solve this problem by converting these unpleasant sounding vibrations into musical tones. To do so, the controller 20 is configured to synchronize vibration frequencies generated by the vibration motors 18 with a selected musical score to generate musical tones, and then output these generated musical tones for the user to hear instead of the vibration noises. The buttons 22, 24 are disposed on a single board or console 30, with a “short” press button 22 being encircled by a “long” press button 24. For example, the short-press button 22, when engaged (i.e., pressed) by the user, controls the controller 20 to cycle through musical score presets (stored in the memory 32) until a user disengages the short press-button 22 to select a musical score. The long press button 24, when engaged by the user, controls the controller 20 to adjust vibration amplitude levels of the vibration motors 18.

To generate the musical tones, the controller 20 is configured to perform a pulse width modulation (PWM) process, with instructions to do so stored in the memory 32 and executed by the electronic processor 34. In some embodiments, the PWM process can be a sinusoidal PWM (SPWM) process to modulate vibration frequencies of one or more of the vibration motors 18 to match musical notes. The memory 32 of the controller 20 stores a table 36 that includes predetermined musical scores matched to predetermined vibration frequencies. The table can be stored in non-volatile RAM or other computer memory.

In one embodiment, the user (wearer) can operate the short press button 22 to select a musical score stored in the non-volatile memory of the controller 20. The controller 20 is configured to access the table 36 to derive the frequencies corresponding to the musical notes of the selected musical score, and the controller 20 then drives the motors 18 in a controlled sequential manner at select frequencies that correspond to the musical notes of the musical score such that the sounds generated by the vibration of the motors 18 is perceived (heard) by the user (wearer) as the selected musical score. Alternatively, the device 10 can also include one or more audio speakers that can output audible musical tones of the selected musical score that match the frequencies at which the vibration motors 18 are driven, in a synchronized manner therewith, such that the vibrations from the motors 18 are synchronized with audiable musical tones output by the speaker(s).

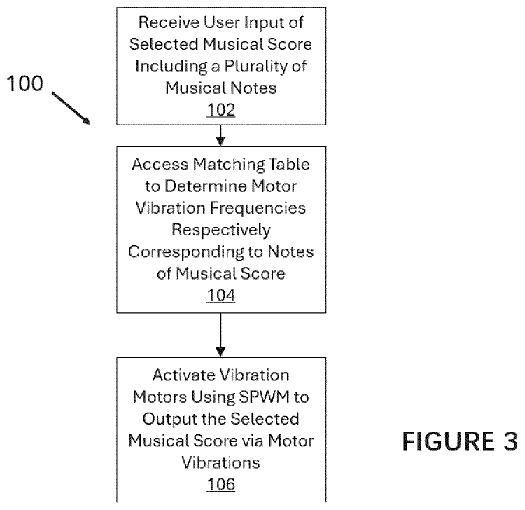

FIG. 3 shows a flow chart of operations of a massage method 100 that can be performed by the massage device 10 to generate the musical tones from the mechanical vibrations of the vibration motors 18. To begin the method 100, a user can turn on the backpack 10 using a power button. The short press button 22 and/or long press button 24 can be configured to operate as a power on/off button, e.g., by a user pressing both buttons 22,24 simultaneously and/or by a user pressing and holding one and/or both buttons 22,24 for an extended period of time such as 3 seconds or more.

In an operation 102, the user engages the short press button 22 to select a stored musical score and can optionally use the long press button 24 to adjust an amplitude level of the vibration motors 18.

In an operation 104, the controller 20 accesses a matching table 36 (described below) to determine a plurality of motor vibration frequencies respectively corresponding to notes of the selected musical score.

In an operation 106, the controller 20 activates the two or more vibration motors 18 in a controlled synchronized manner to vibrate at the motor vibration frequencies respectively corresponding to notes of the selected musical score such that the musical score is output to a user by way of the vibrations of the vibration motors 18, while the vibrations of the vibration motors are simultaneously used to provide a massage to the user/wearer of the device 10. In this manner, the musical score is output to the user/wearer of the device 10 in the form of vibration tones output by the vibration motors 18 while the user receives a massage from the vibration motors 18.

The controller 20 is programmed to implement a SPWM process to generate sinusoidal waveforms matching musical frequencies. For example, the SPWM process can convert musical notes into corresponding pulse-width signals to drive the vibration motors 18 at select frequencies corresponding to respective musical notes. Advantageously, irregular vibrations from the vibration motors 18 are replaced with structured sinusoidal waves, eliminating broadband noise.

Although not required, an optional audio output speaker can also be provided on the device 10 to output the musical score in a synchronized manner to match the vibrations of the motors 18.

When the user is finished using the backpack 10, the user can press a power on/off switch, which will turn off both the vibration motors 18 and the musical notes.

In some embodiments, the vibration frequencies of the vibration motors 18 can be dynamically adjusted to corresponding to musical scales to convert the noise from the vibration frequencies of the vibration motors 18 into the selected musical notes or melodies. For example, if the user presses the short press button 22 to select a different musical scale, the controller 20 can control the vibration motors 18 to generate vibration frequencies that match the new musical scale or notes selected by the user, wherein such frequencies corresponding to different musical scales can be stored in the matching table 36 and accessed by the controller 20. The controller 20 can be programmed to adjust a duty cycle of the generated vibration frequencies for each vibration motor 18 to ensure the selected musical score continues to be output. The adjustment of the duty cycle of the SPWM signals of the vibration frequencies regulates vibration intensity while maintaining musical fidelity.

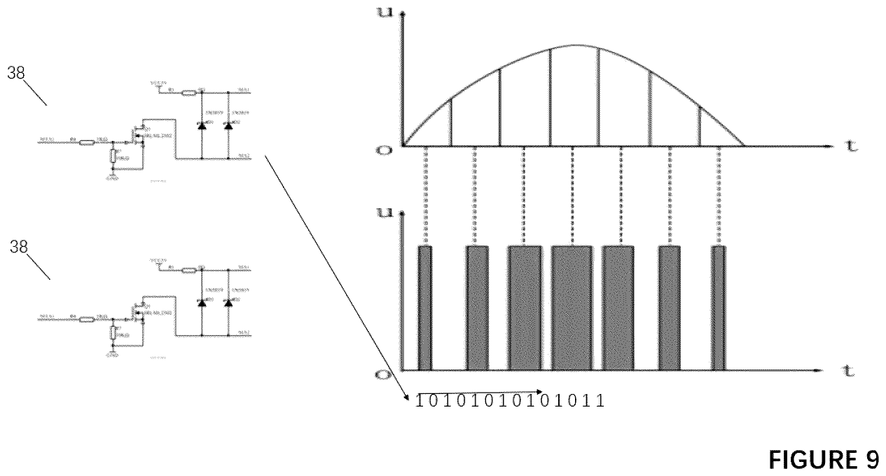

In some embodiments, the controller 20 is optionally programmed to independently and separately control a vibration frequency of each vibration motor 18. Advantageously, this independent control allows for synchronized music generation by the controller 20 wherein the vibrations of the motors 18 generate the sound perceived by the user/wearer of the device 10. In this manner, the vibrating sounds output by the vibrating motors 18 are experienced and perceived by a user/wearer of the device 10 to be the musical score selected by the user/wearer. In one example, the controller 20 includes metal-oxide-semiconductor field-effect transistor (MOSFET) circuits (shown in FIG. 9) corresponding to the number of vibration motors 18, wherein each MOSFET circuit controls operation of a corresponding respective vibration motor 18.

The controller 20 can include other typical computer processing components to perform the method 100. For example, the electronic processor 34 can implement a main computing unit (MCU) (not shown) to generate the SPWM signals and process user inputs provided via the buttons 22, 24. In another example, the electronic processor 34 can include one or more filtering circuit (not shown) to stabilize voltage and reduce electromagnetic interference of the vibration motors 18.

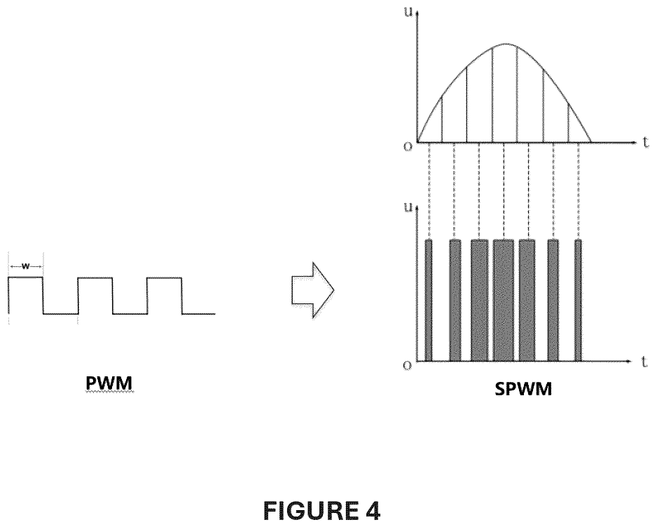

FIGS. 4-9 show an example process of generating the musical tones from the vibration frequencies. The controller 20 is configured to perform a pulse width modulation (PWM) process, with instructions to do so stored in the memory 32 and executed by the electronic processor 34. In some embodiments, the PWM process can be a sinusoidal PWM (SPWM) process. FIG. 3 shows a graph showing how the SPWM process works. The SPWM process makes an output current or voltage of the vibration motors 18 as close to sinusoidal as possible to power the vibration motors 18. The sinusoidal graph of FIG. 4 shows a sine wave of equal width and unequal amplitude, and the corresponding bar graph of FIG. 4 shows rectangular pulses of equal amplitude and unequal width. The SPWM process is performed for precise control of control signals generated by the controller 20 to achieve a descried vibration profile.

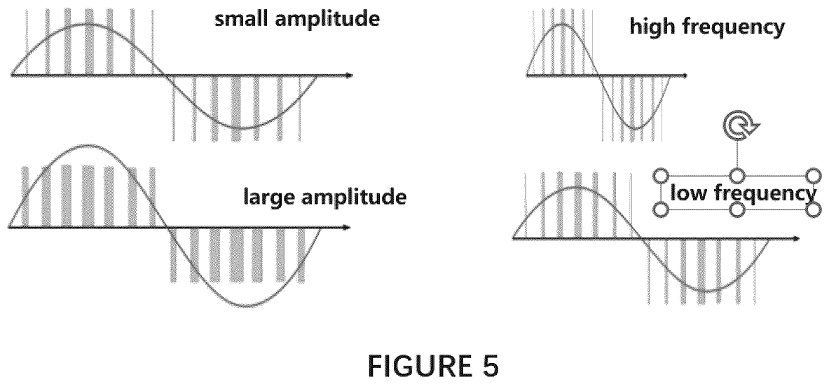

As shown in FIG. 5, four sinusoidal graphs are shown with corresponding rectangular pulses needed to achieve the desired sinusoidal wavelength for small amplitude control signals, large amplitude control signals, high frequency control signals, and low frequency control signals.

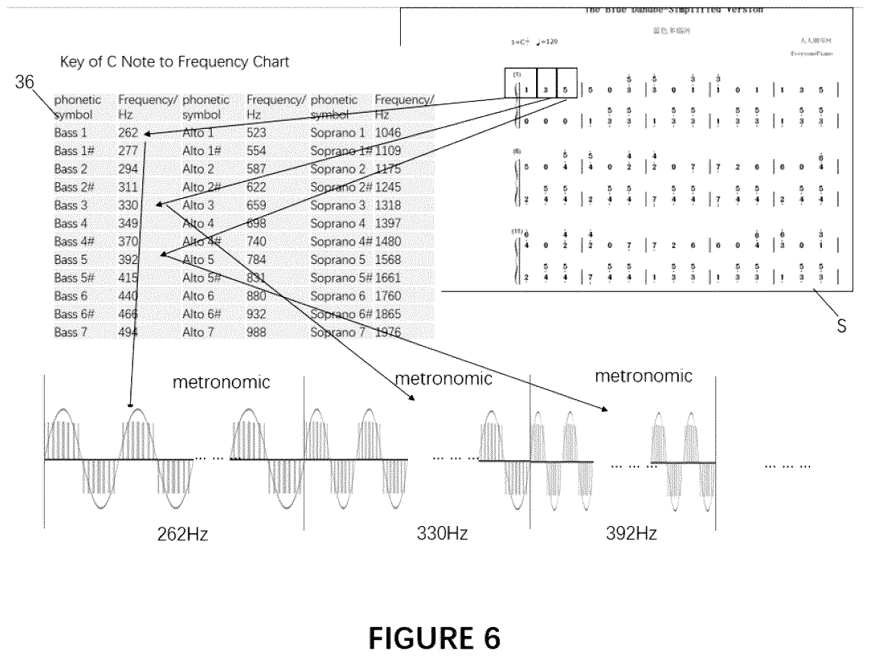

FIGS. 6-8 show an example of the matching operation 106 of the method 100. FIG. 6 shows an example of a matching table 36 stored in the memory of the controller 20. The table 36 can include columns with different frequency ranges or “scales” (“bass”, “alto” and “soprano” are shown in the illustrated example table 36. Each frequency range or scale comprises a plurality of distinct frequency notes labeled 1,1 #,2,2 #,3,4,4 #,5,5 #,6,6 #,7 which correspond respectively to a particular musical note. The desired scale (Bass, Alto, Soprano) can be selected by the user using the short press button 22. Each scale includes a corresponding frequency, ranging from 262 Hz to 494 Hz for the bass scale, 523 Hz and 988 Hz for the alto scale, and 1046 Hz to 1976 Hz for the soprano scale. A musical composition or score S with a preselected melody is also stored in the memory 32 and used with the table 36. The musical score S comprises a plurality of musical notes, wherein each note corresponds to one of the frequency notes stored in the table 36 for each scale of the table. The illustrated score S comprises, for example, three notes in the first bar (labeled as “1”, “3” and “5” in the sheet S) corresponding to different controlled frequency vibrations of the vibration motors 18. These notes correspond to the corresponding controlled vibration frequencies of the motors 18. For example, as shown in FIG. 6, note “1” corresponds to a motor vibration frequency of 262 Hz in the Bass scale, note “3” corresponds to a motor vibration frequency of 330 Hz in the Bass scale, and note “5” corresponds to a motor vibration frequency of 392 Hz in the Bass scale (the notes “1” “3” “5” correspond to other respective motor vibration frequencies in the Alto scale and in the Soprano scale). Also shown in FIG. 6 are the graphical SPWM signals generated by the controller 20 to drive the motors 18 at frequencies corresponding to these three notes for the selected Bass scale. Those of ordinary skill in the art will recognize that the Alto scale or the Soprano scale could alternatively be selected by a user, in which case the notes “1” “3” and “5” (and the other notes) have different respective corresponding motor vibration frequencies as listed in the matching table 36.

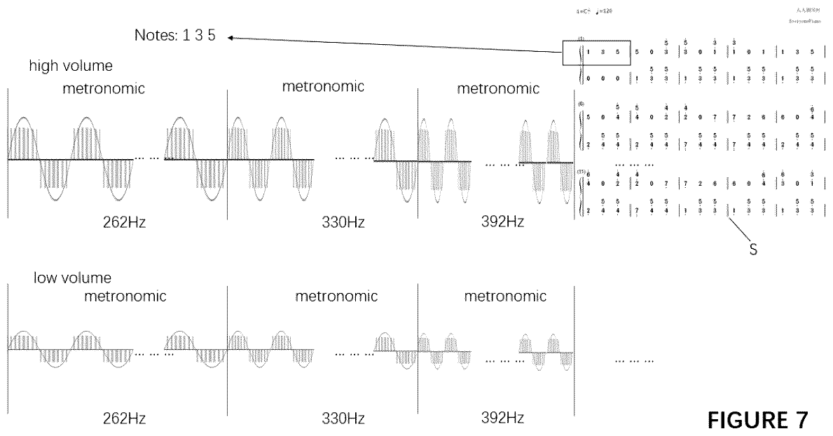

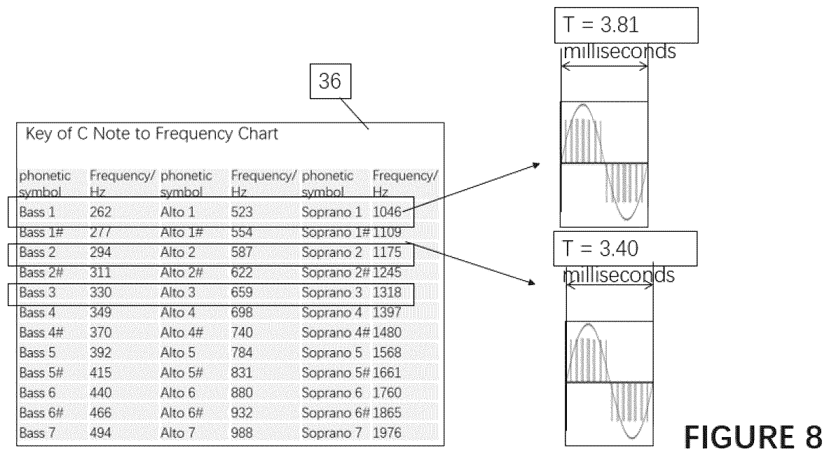

FIG. 7 shows the SPWM signals for the three notes for a high volume and a low volume (selected by the user using the long press button 24). FIG. 8 shows that the duty cycle of the generated vibration frequencies can be adjusted (e.g., showing a frequency period of 3.81 milliseconds for a frequency of 262 Hz, and a frequency period of 3.40 milliseconds for a frequency of 294 Hz).

FIG. 9 shows an example of the independent control of each vibration motor 18 by the controller 20. Two MOSFET motor drive circuits 38 are shown in FIG. 9. Each motor drive circuit 38 is operatively connected to the controller 20 and operatively connected to a respective vibration motor 18 and controls the vibration motor 18 to be active or inactive depending upon if the circuit 38 outputs a value of “1” to supply energy to the vibration motor 18 (corresponding to the associated controlled motor 18 being active to generate a vibration tone) or if the circuit outputs a value of “0” to stop the flow of energy to the vibration motor 18 (corresponding to the associated controlled motor 18 to be inactive so as not to output any vibration tone).

One of ordinary skill in the art will recognize that the present disclosure provides a vibration noise-to-music conversion system for a massage device. The conversion system includes a body; at least one massage device disposed on the body, the at least massage device including at least two vibration motors; and a controller programmed to execute a Sinusoidal Pulse Width Modulation (SPWM) algorithm, wherein the SPWM algorithm modulates vibration frequencies of the at least two vibration motors to correspond to musical notes. The controller synchronizes the vibration frequencies of the at least two vibration motors with a selected musical score to generate structured musical tones through the vibrations of the motors 18. Advantageously, irregular vibrations from the vibration motors 18 are replaced with structured sinusoidal waves, eliminating broadband noise. In some embodiments, a user interface is configured for selecting musical scores and adjusting vibration amplitude. In some embodiments, the SPWM algorithm generates sinusoidal waveforms by varying pulse widths to match frequencies of musical scales. In some embodiments, the at least two vibration motors are independently driven by MOSFET-based circuits to enable zone-specific frequency control. In some embodiments, the user interface includes short-press buttons for cycling through musical presets; long-press buttons for adjusting vibration amplitude levels. In some embodiments, the controller is programmed to filter electromagnetic interference from motor driver circuits to maintain music signal fidelity. In some embodiments, the SPWM algorithm dynamically adjusts pulse duty cycles to control both frequency and amplitude.

One of ordinary skill in the art will recognize that the present disclosure provides a method for converting vibration noise into musical tones that includes generating vibrations via at least three motors, modulating the vibration frequencies of the motors using an SPWM algorithm to align with musical note frequencies, receiving user input to select a musical score and adjust vibration intensity, and synchronizing the modulated frequencies across the motors to produce coherent musical output. In some embodiments, the method includes selecting, via a user interface, musical scores and adjusting vibration amplitude. In some embodiments, the SPWM algorithm generates sinusoidal waveforms by varying pulse widths to match frequencies of musical scales. In some embodiments, the at least three vibration motors are independently driven by MOSFET-based circuits to enable zone-specific frequency control. In some embodiments, the user interface includes short-press buttons for cycling through musical presets; long-press buttons for adjusting vibration amplitude levels. In some embodiments, the method includes filtering electromagnetic interference from motor driver circuits to maintain music signal fidelity. In some embodiments, the SPWM algorithm dynamically adjusts pulse duty cycles to control both frequency and amplitude.

One of ordinary skill in the art will recognize that the present disclosure provides a massage device that includes a main body. At least two vibration motors are disposed within the main body. A controller is programmed to synchronize vibration frequencies generated by the at least two vibration motors with a selected musical score to generate musical tones; and output the generated musical tones. In some embodiments, the massage device includes a user interface that includes a short-press button that, when engaged by a user, is configured to control the controller to cycle through musical score presets until a user disengages with the short press-buttons to select the selected musical score, and a long-press button that, when engaged by a user. is configured to control the controller to adjust vibration amplitude levels of the at least two vibration motors. In some embodiments, the controller is further programmed to adjust a duty cycle of the generated vibration frequencies to ensure the selected musical score continues to be output. In some embodiments, the main body comprises a backpack comprising a storage portion and two shoulder straps. The at least two vibration motors comprise three vibration motors, a first vibration motor being disposed in a first shoulder strap, a second vibration motor being disposed in a second shoulder strap, and a third vibration motor being disposed in the storage portion.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

1. A vibration noise-to-music conversion system for a massage device, the conversion system comprising:
a body;
at least one massage device disposed on the body, the at least massage device including at least two vibration motors;
a controller programmed to execute a Sinusoidal Pulse Width Modulation (SPWM) algorithm, wherein the SPWM algorithm modulates vibration frequencies of the at least two vibration motors to correspond to musical notes;
wherein the controller synchronizes the vibration frequencies of the at least two vibration motors with a selected musical score to generate structured musical tones.

2. The system of claim 1, further including a user interface configured for selecting musical scores and adjusting vibration amplitude.

3. The system of claim 1, wherein the SPWM algorithm generates sinusoidal waveforms by varying pulse widths to match frequencies of musical scales.

4. The system of claim 1, wherein the at least two vibration motors are independently driven by MOSFET-based circuits to enable zone-specific frequency control.

5. The system of claim 2, wherein the user interface includes short-press buttons for cycling through musical presets; long-press buttons for adjusting vibration amplitude levels.

6. The system of claim 1, wherein the controller is programmed to filter electromagnetic interference from motor driver circuits to maintain music signal fidelity.

7. The system of claim 1, wherein the SPWM algorithm dynamically adjusts pulse duty cycles to control both frequency and amplitude.

8. A method for converting vibration noise into musical tones, comprising:
generating vibrations via at least three motors;
modulating the vibration frequencies of the motors using an SPWM algorithm to align with musical note frequencies;
receiving user input to select a musical score and adjust vibration intensity;
synchronizing the modulated frequencies across the motors to produce coherent musical output.

9. The method of claim 8, further including selecting, via a user interface, musical scores and adjusting vibration amplitude.

10. The method of claim 8, wherein the SPWM algorithm generates sinusoidal waveforms by varying pulse widths to match frequencies of musical scales.

11. The method of claim 8, wherein the at least three vibration motors are independently driven by MOSFET-based circuits to enable zone-specific frequency control.

12. The method of claim 9, wherein the user interface includes short-press buttons for cycling through musical presets; long-press buttons for adjusting vibration amplitude levels.

13. The method of claim 9, further comprising filtering electromagnetic interference from motor driver circuits to maintain music signal fidelity.

14. The method of claim 8, wherein the SPWM algorithm dynamically adjusts pulse duty cycles to control both frequency and amplitude.

15. A massage device, comprising:
a main body;
at least two vibration motors disposed within the main body;
a controller programmed to:
synchronize vibration frequencies generated by the at least two vibration motors with a selected musical score to generate musical tones; and
output the generated musical tones.

16. The massage device of claim 15, further comprising a user interface that includes:
a short-press button that, when engaged by a user, is configured to control the controller to cycle through musical score presets until a user disengages with the short press-buttons to select the selected musical score; and
a long-press button that, when engaged by a user is configured to control the controller to adjust vibration amplitude levels of the at least two vibration motors.

17. The massage device of claim 15, wherein the controller is further programmed to:
adjust a duty cycle of the generated vibration frequencies to ensure the selected musical score continues to be output.

18. The massage device of claim 15, wherein the main body comprises a backpack comprising a storage portion and two shoulder straps;
wherein the at least two vibration motors comprise three vibration motors, a first vibration motor being disposed in a first shoulder strap, a second vibration motor being disposed in a second shoulder strap, and a third vibration motor being disposed in the storage portion.
